(19) United States
(12) Patent Application Publication
(10) Pub. No.: US 2005/0222100 A1
(43) Pub. Date: Oct. 6, 2005

(54) Treatment of post-menopausal complaints in breast cancer patients comprising tibolone and a serm

(76) Inventors: H. J. Kloosterboer, Oss (NL); A. E. P. Adang, Oss (NL)

Correspondence Address: AKZO NOBEL INC.;INTELLECTUAL PROPERTY DEPARTMENT, 7 LIVINGSTONE AVENUE, DOBBS FERRY, NY, 10522-3408, US

(21) Appl. No.: 10/515,712

(22) PCT Filed: May 20, 2003
(86) PCT No.: PCT/EP03/50178

§ 371 Date: Nov. 23, 2004

(30) Foreign Application Priority Data
May 24, 2002 (EP) 02077050.9

(51) Int. Cl. A61K 31/57; A61K 31/445; A61K 31/138

(52) U.S. Cl. 514/171; 514/319; 514/651

(57) ABSTRACT

The subject invention provides a use of tibolone and a SERM for the manufacture of a medicine for the treatment of an estrogen-deficiency related complaint and for the prevention of a recurrence of breast cancer in females suffering from, or at risk for breast cancer that exhibit the estrogen-deficiency related complaint.

### FIELD OF THE INVENTION

The subject invention concerns female cancer patients on treatment with selective estrogen receptor modulators (SERM's).

### BACKGROUND

Female breast cancer patients or female patients at risk for breast cancer, being treated with selective estrogen receptor modulator (SERM) anti-cancer drugs suffer from estrogen deficiency related complaints.

SERM's cause estrogen-deficiency related complaints as a result of their action at the level of the estrogen receptors. SERM's do not however actively suppress the endogenous estrogen synthesis. Therefore women on treatment with SERM's still have some circulating estrogens (formed from precursors produced by the adrenals) who's action is subject to competition by estrogen receptor antagonism. This is unlike other anti-cancer drugs such as aromatase inhibitors, 17β-hydroxy steroid dehydrogenase inhibitors and sulfatase inhibitors which act on the metabolic pathway which leads to the synthesis of endogenous estrogens, thereby actively suppressing the synthesis of endogenous estrogens.

Common SERM's used in anti-cancer treatment are tamoxifen (a partial estrogen receptor antagonist) and raloxifene (a selective estrogen receptor modulator).

Estrogen-deficiency related complaints, such as climacteric complaints and bone loss, are also well-known as symptoms in (post) menopausal women. For these illnesses and symptoms, various treatments exist, such as estradiol supplementation, combination of estrogens and progestagens, and other drugs.

However, the existing treatments for post-menopausal women are not suitable for women which suffer, or have suffered from, breast cancer or are known to have a risk for breast cancer. The reason is that the typical drugs used for estrogen-supplementation will increase the recurrence of, or even cause, breast tumors. In fact, it is one of known effects of estrogens and estrogen-like therapies that they stimulate breast (mammary glands) and uterus.

The compound tibolone, (7α, 17α)-17-hydroxy-7-methyl-19-nor-17-pregn-5(10)-en-20-yn-3-one, is known as a tissue-specific and effective agent that can be used in hormone replacement therapy (HRT) in (post) menopausal women, for the treatment of menopausal and postmenopausal disorders, including climacteric complaints, vasomotor symptoms, osteoporosis, and vaginal atrophy (U.S. Pat. No. 5,037,817, WO 98/47517).

Tibolone, also known as Livial®, is a synthetic compound, which shows weak estrogenic, androgenic and progestagenic activities compared to estrogen, progesterone, and androgen receptors. Previous studies have shown favorable effects on bone, the vagina, the cardiovascular system, climacteric symptoms, mood, and libido without detrimental estrogen-like stimulation of the breast and endometrium (Kloosterboer, 2001; Kloosterboer et al., 2000; Pain Research and Nuffield Department of Anaesthetics, 1999; Tang et al., 1993). Studies have indicated that tibolone increases bone mineral density (BMD) relative to baseline or placebo over periods ranging from six months to three years (Pain Research and Nuffield Department of Anaesthetics, 1999).

Tibolone, at any rate prior to this invention, is subject to a warning for use in cancer-endangered patients. EP 613687 describes tibolone for the prevention or treatment of tumors. However, EP 613687 relates to a different medical indication than that according to the subject invention.

It has now surprisingly been found that in women suffering from, or at risk for breast cancer, the administration of a SERM, such as tamoxife n, in conjunction with tibolone reduces, prevents and/or delays both climacteric symptoms as well as the recurrence of the breast cancer.

This is an unexpected finding, not only because of the inherent difficulty in finding any treatment at all in the above special population, but also because tibolone itself hardly has any estrogenic activity, and is metabolized to compounds which have an approximately fifty-fold lower estrogenic receptor activity than estradiol. That particularly this drug works in the treatment of complaints related to estrogen-deficiency is surprising.

The combined use of tibolone and a SERM, such as tamoxifen, in the special population discussed above has not been disclosed in the art, nor can its favourable and safe activity be derived therefrom.

WO 01/54699 (Endorecherche Inc.) describes the addition of a SERM to estrogen supplementation therapy in post-menopausal women to treat or reduce post-menopausal complaints. WO 01/54699 does not however disclose or suggest the specific use of tibolone (which is not an estrogen) in combination with a SERM for the treatment of the special population of female patients suffering from breast-cancer or at risk thereof.

Tibolone, although mentioned in WO 01/54699 as part of a list with estrogens, is in fact not an estrogen as detailed above and WO 01/54699 fails to show the beneficial effect of tibolone with a SERM for the treatment of post-menopausal complaints. Moreover, WO 01/54699 does not at all relate to the special population of women suffering from breast cancer.

### SUMMARY OF THE INVENTION

The subject invention provides a concomitant use of a pharmaceutically effective amount of tibolone and a pharmaceutically effective amount of a SERM for the manufacture of a medicine for the treatment of an estrogen-deficiency related complaint and for the prevention of a recurrence of breast cancer in females suffering from, or at risk for breast cancer who exhibit the estrogen-deficiency related complaint.

### FIGURES

FIG. 1A: Mean number of hot flushes in women on placebo+tamoxifen vs. women on tibolone+tamoxifen determined by diary card.

FIG. 1B: number of hot flushes in women on placebo+tamoxifen vs. women on tibolone+tamoxifen determined by diary card.

FIG. 2A: Severity of hot flushes in women on placebo+tamoxifen vs. women on tibolone+tamoxifen determined by diary card.

FIG. 2B: Severity of hot flushes in women on placebo+tamoxifen vs. women on tibolone+tamoxifen determined by diary card.

FIG. 3: intensity score of hot flushes in women on placebo+tamoxifen vs. women on tibolone+tamoxifen.

FIG. 4: intensity score of night sweats in women on placebo+tamoxifen vs. women on tibolone+tamoxifen.

FIG. 5: intensity score of hot flushes/night sweats interference in normal life in women on placebo+tamoxifen vs. women on tibolone+tamoxifen.

FIG. 6: intensity score of vaginal dryness in women on placebo+tamoxifen vs. women on tibolone+tamoxifen.

FIG. 7: irregular vaginal bleeding in women on placebo+tamoxifen vs. women on tibolone+tamoxifen.

FIG. 8: endometrial thickness in mm in women on placebo+tamoxifen vs. women on tibolone+tamoxifen.

FIG. 9: endometrial thickness in % change from baseline in women on placebo+tamoxifen vs. women on tibolone+tamoxifen.

### DETAILED DESCRIPTION OF THE INVENTION

The subject invention provides a use of a pharmaceutically effective amount of tibolone and a pharmaceutically effective amount of a SERM for the manufacture of a medicine for the treatment of an estrogen-deficiency related complaint and for the prevention of a recurrence of breast cancer in females suffering from, or at risk for breast cancer who exhibit the estrogen-deficiency related complaint.

The subject invention further provides a method of treating an estrogen-deficiency related complaint in a female patient suffering from, or at risk for a breast cancer that exhibits the complaint, wherein the treatment comprises the administration to said patient of a pharmaceutically effective amount of tibolone in conjunction with a pharmaceutically effective amount of a SERM, together effective to treat the complaint and to prevent recurrence of the breast cancer.

The subject invention also contemplates a kit for treating an estrogen-deficiency related complaint in a female patient suffering from, or at risk for a breast cancer comprising a first container comprising a therapeutically effective amount of tibolone and a second container comprising a therapeutically effective amount of a SERM.

The SERM used in the subject invention can be any SERM known in the art. More specifically, the SERM can be selected from the group consisting of tamoxifen, 4-hydroxy tamoxifen, raloxifene, EM-800, EM-652.HCl, arzoxifene (LY 353 381), LY 335 563, GW-5638, Lasofoxifene, bazedoxifene (TSE 424) and prodrugs thereof. In a preferred embodiment, the SERM is tamoxifen. In another embodiment, the SERM is raloxifene.

In one embodiment, the estrogen-deficiency related complaint encompasses a climacteric complaint.

More specifically, the climacteric complaint encompasses hot flushes, night sweats, vaginal dryness, and any other known climacteric symptom.

In another embodiment, the estrogen-deficiency related complaint encompasses bone loss.

Tibolone and the elected SERM can be administered by any known route of administration. Specifically, the administration can be enterally, parenterally, or via implant.

The daily dosage of tibolone is 0.003-3.0 mg per kg body weight; preferably a daily dosage of 0.03-0.4 mg per kg body weight is administered. More preferably, the invention can be carried out by providing tibolone in daily dosage amounts of from 0.2 to 5 mg, preferably 0.3 to 2.5 mg and more preferably fixed dosages of 1.25 or 2.5 mg.

The daily dosage of the SERM, e.g. tamoxifen or raloxifene, is 10-100 mg. In a preferred embodiment, the dosage is 60 mg. In another preferred embodiment, the dosage is 30 mg. In yet another preferred embodiment, the daily dosage is 20 mg.

Mixed with pharmaceutically suitable auxiliaries, e.g. as described in the standard reference, Gennaro et al., Remington's Pharmaceutical Sciences, (18th ed., Mack Publishing Company, 1990, see especially Part 8: Pharmaceutical Preparations and Their Manufacture) the compound may be compressed into solid dosage units, such as pills, tablets, or be processed into capsules or suppositories. By means of pharmaceutically suitable liquids the compound can also be applied as an injection preparation in the form of a solution, suspension, emulsion, or as a spray, e.g. a nasal spray. For making dosage units, e.g. tablets, the use of conventional additives such as fillers, colorants, polymeric binders and the like is contemplated. In general, any pharmaceutically acceptable additive which does not interfere with the function of the active compound can be used.

Suitable carriers with which the compositions can be administered include lactose, starch, cellulose derivatives and the like, or mixtures thereof, used in suitable amounts.

The term container as used herein encompasses any form of pharmaceutical package unit known in the art, e.g. blisters, bottles, sachets, boxes etc. Also a blister in a blister package can be considered a container.

An example of a tablet of tibolone has the following composition:

| | | |
|---|---|---|
| tibolone | 2.5 | mg |
| starch | 10 | mg |
| ascorbyl palmitate | 0.2 | mg |
| magnesium stearate | 0.5 | mg |
| lactose | to make up to 100 | mg |

and is made from base granules prepared by mixing the lactose with a portion of the starch. The remainder of the starch is mixed to a slurry with water and added to the mixture. The whole is granulated and dried. These base granules are mixed with ascorbyl palmitate and tibolone, sieved, finely mixed with magnesium stearate and then tabletted.

### EXAMPLE

A double-blind, randomized, placebo controlled pilot study was carried out in 64 post-menopausal women on treatment with tamoxifen after surgery for early breast cancer.

The women, all below the age of 65 years, were postmenopausal for at least three years at the time of diagnosis. Their follicle stimulating hormone (FSH) levels were greater than 40 IU/L and their estradiol (E₂) levels were below 20 pg/mL. They all had a uterus, normal smear, BMI of 18-29 kg/m², no other malignancy or serious disease and smoked less than 10 cigarettes per day.

The women were divided into two groups of 32 women:
I. 2.5 mg tibolone (Livial®) per day and 20 mg tamoxifen (Nolvadex-D®) per day for 12 months
II. placebo and 20 mg/day tamoxifen (Nolvadex-D®) for 12 months

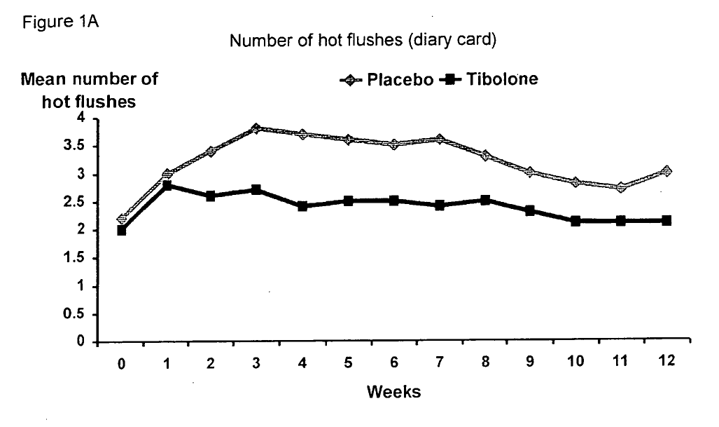

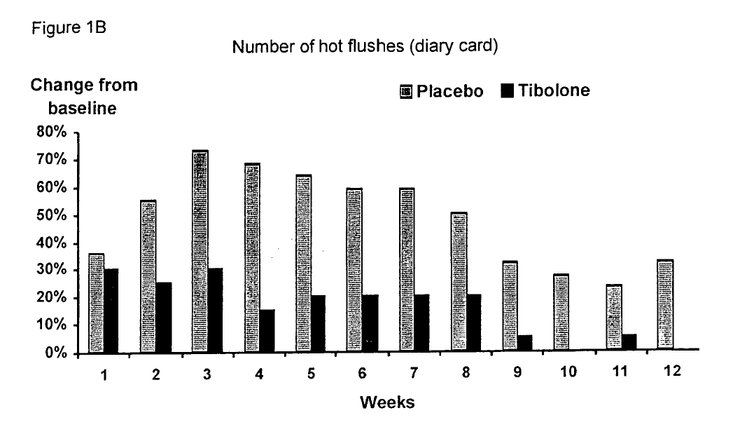

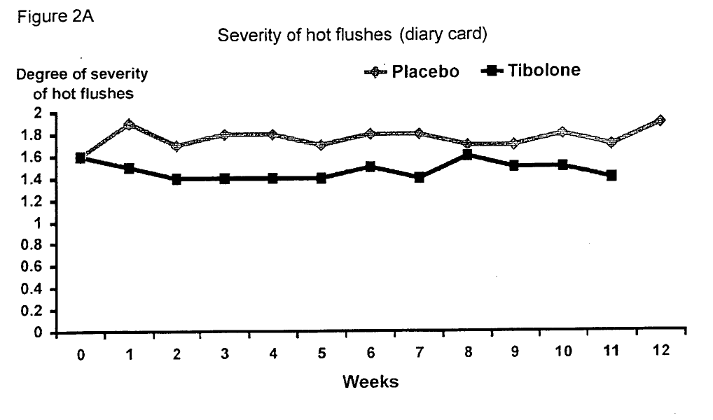

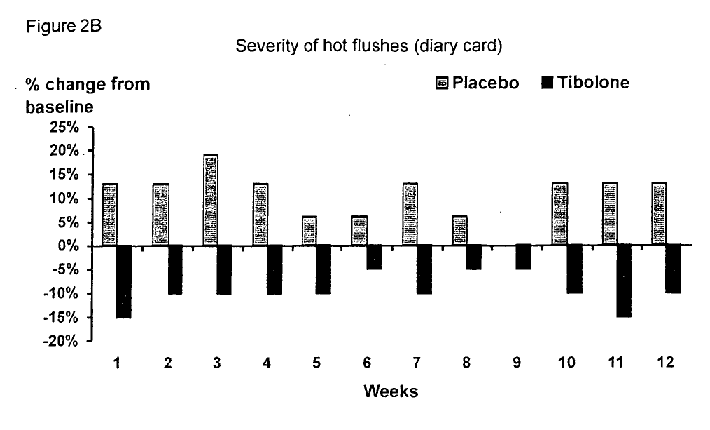

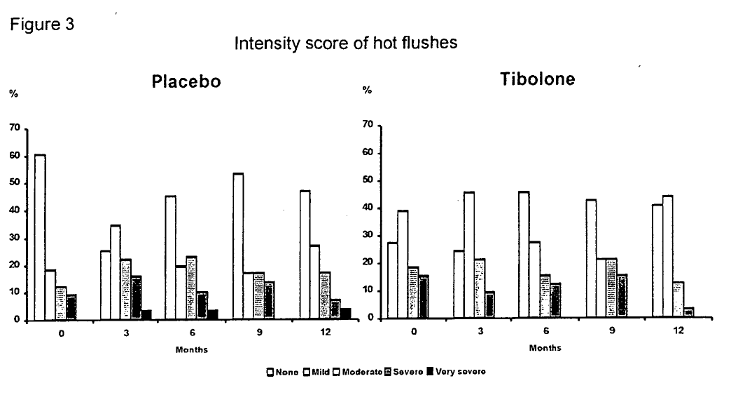

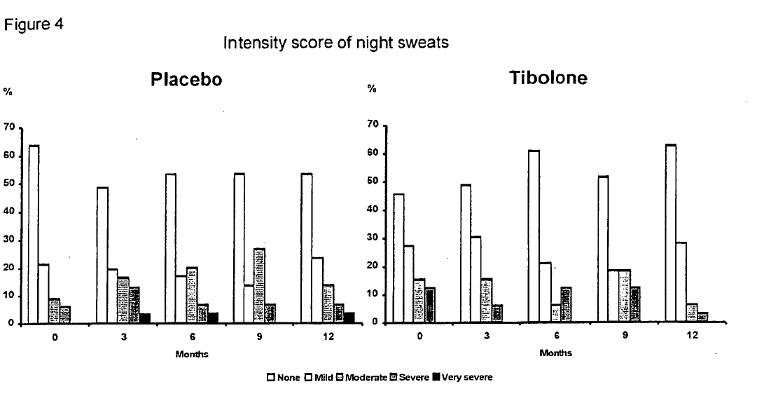

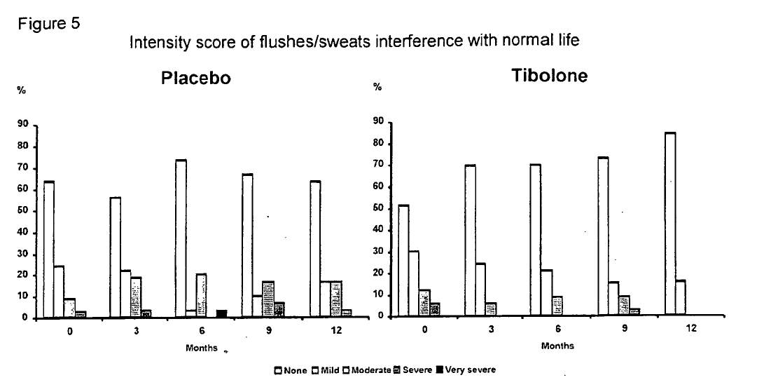

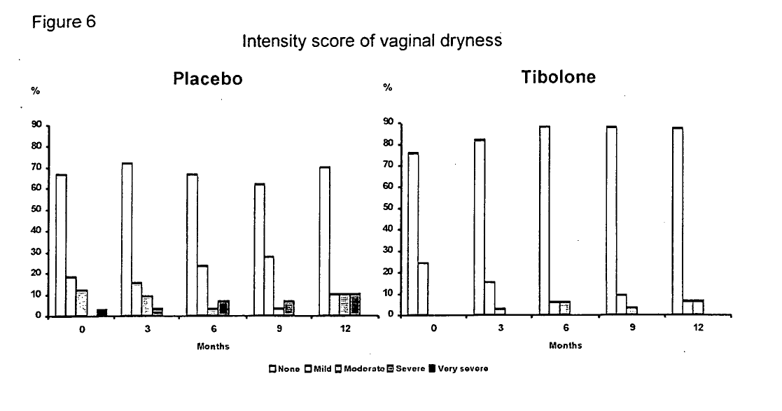

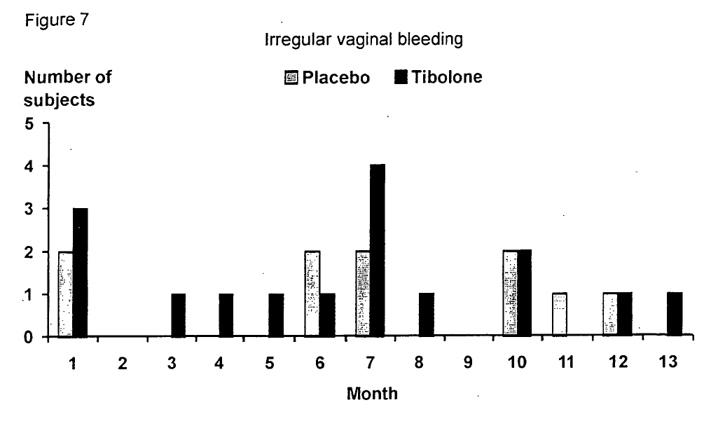

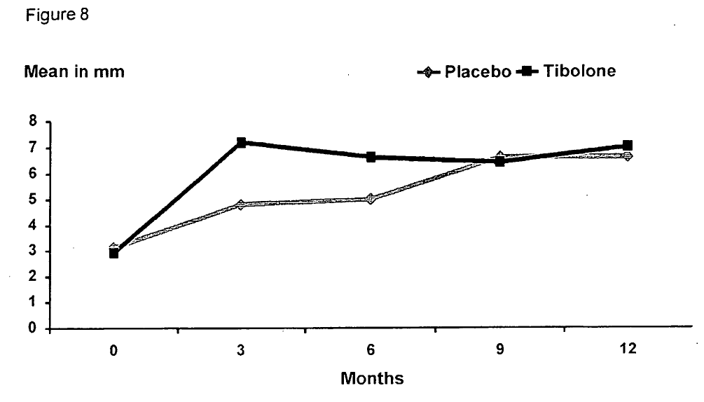

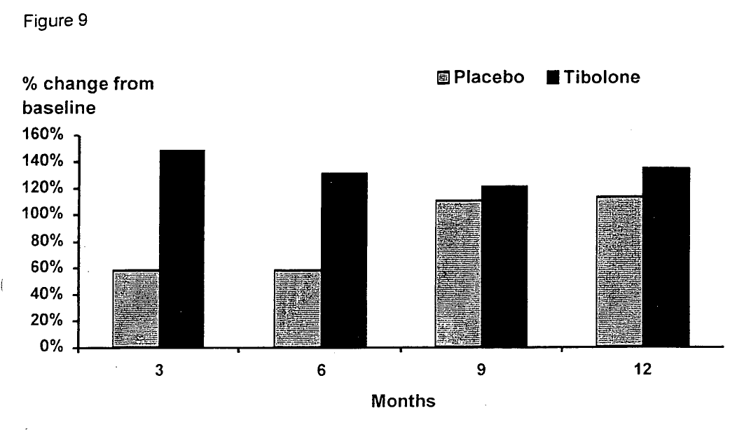

The results show (FIGS. 1-9) that all climacteric symptoms tested, i.e. hot flushes, night sweats, and vaginal dryness improved in women taking tibolone and tamoxifen as opposed to placebo and tamoxifen. Tibolone had minimal effect on irregular bleeding.

Endometrial thickness was measured by means of transvaginal ultrasound. Tibolone had a similar effect as placebo after 9 and 12 months on endometrial thickness. Thus, tibolone may prevent and neutralize endometrial stimulation associated with tamoxifen administration.

Endometrial biopsies were taken after 6 and 12 months. No clinically significant effect on endometrial histology was observed after 12 months. This positive result is surprising in view of the fact that tamoxifen is known to have a negative influence on the endometrium.

Moreover, no recurrence of breast cancer occurred in any of the women tested.

1. (canceled)

2. (canceled)

3. (canceled)

4. (canceled)

5. (canceled)

6. (canceled)

7. (canceled)

8. (canceled)

9. (canceled)

10. A method of treating an estrogen-deficiency related complaint in a female patient suffering from, or at risk for a breast cancer, comprising:
administering a pharmaceutically effective amount of tibolone in conjunction with a pharmaceutically effective amount of a selective estrogen receptor modulator (SERM), together effective to treat the complaint and to prevent recurrence of the breast cancer.

11. The method according to claim 10, wherein the SERM is tamoxifen.

12. The method according to claim 10, wherein the SERM is raloxifene.

13. The method according to claim 10, wherein the estrogen-deficiency related complaint comprises a climacteric complaint.

14. The method according to claim 13, wherein the climacteric complaint comprises hot flushes.

15. The method according to claim 13, wherein the climacteric complaint comprises night sweats.

16. The method according to claim 13, wherein the climacteric complaint is vaginal dryness.

17. The method of claim 10, wherein the estrogen-deficiency related complaint comprises bone loss.

18. The method of claim 10, wherein tibolone is administered in a daily dosage of 0.3 to 2.5 mg.

19. A kit for treating an estrogen-deficiency related complaint in a female patient suffering from, or at risk for a breast cancer, comprising:
a first container comprising a therapeutically effective amount of tibolone and
a second container comprising a therapeutically effective amount of a SERM.

20. The kit according to claim 19, wherein the SERM is tamoxifen.

21. The kit according to claim 19, wherein the SERM is raloxifene.

22. The kit according to claim 19, wherein the estrogen-deficiency related complaint comprises a climacteric complaint.

23. The kit according to claim 22, wherein the climacteric complaint comprises hot flushes.

24. The kit according to claim 22, wherein the climacteric complaint comprises night sweats.

25. The kit according to claim 22, wherein the climacteric complaint is vaginal dryness.

26. The kit according to claim 19, wherein the estrogen-deficiency related complaint comprises bone loss.

27. The kit according to claim 19, wherein tibolone is administered in a daily dosage of 0.3 to 2.5 mg.
